(19) United States
(12) Patent Application Publication
(10) Pub. No.: US 2023/0130064 A1
(43) Pub. Date: Apr. 27, 2023

(54) FORM-IN-PLACE SHAPE-STABILIZED PHASE CHANGE MATERIAL FOR TRANSIENT COOLING

(71) Applicant: GE Precision Healthcare LLC, Wauwatosa, WI (US)

(72) Inventors: Naveenan Thiagarajan, Clifton Park, NY (US); Warren Lee, Niskayuna, NY (US)

(21) Appl. No.: 17/509,593

(22) Filed: Oct. 25, 2021

(51) Int. Cl. F28F 3/12; A61B 8/00

(52) U.S. Cl. CPC F28F 3/12; A61B 8/546

(57) ABSTRACT

Systems and methods are provided for form-in-place shape-stabilized phase change material for transient cooling. An example combined structure, in accordance with the present disclosure, may include a heat spreading structure and a material structure for holding phase change material. The material structure may be arranged or disposed on a surface of the heat spreading structure. The phase change material may be configured to change phase in response to heat from a component of a system that includes the combined structure, and the material structure has mechanical and structural characteristics for holding the phase change material within the material structure when the phase change material changes phase. The phase change material may be mixed with filler material and/or additives for further assisting in holding the shape of phase change material within the material structure when the phase change material changes phase.

### CROSS-REFERENCE TO RELATED APPLICATIONS

This patent application is related to U.S. patent application Ser. No. 16/212,863, filed Dec. 7, 2018. The above identified application is hereby incorporated herein by reference in its entirety.

### FIELD

Aspects of the present disclosure relate to medical imaging solutions. More specifically, certain embodiments relate to methods and systems for form-in-place shape-stabilized phase change material for transient cooling.

### BACKGROUND

Various medical imaging techniques may be used, such as in imaging organs and soft tissues in a human body. Examples of medical imaging techniques include ultrasound imaging, computed tomography (CT) scans, magnetic resonance imaging (MRI), etc. The manner by which images are generated during medical imaging depends on the particular technique.

For example, ultrasound imaging uses real time, non-invasive high frequency sound waves to produce ultrasound images, typically of organs, tissues, objects (e.g., fetus) inside the human body. Images produced or generated during medical imaging may be two-dimensional (2D), three-dimensional (3D), and/or four-dimensional (4D) images (essentially real-time/continuous 3D images). During medical imaging, imaging datasets (including, e.g., volumetric imaging datasets during 3D/4D imaging) are acquired and used in generating and rendering corresponding images (e.g., via a display) in real-time.

In some instances, medical imaging systems may incorporate heat management functions, which may be configured to maintaining or ensuring particular thermal characteristics during operation of the medical imaging systems. Such heat management functions may require use of particular material, such as phase change material (PCM). Existing solutions for handling use of PCM may have various limitations and disadvantages. Further limitations and disadvantages of conventional and traditional approaches will become apparent to one of skill in the art, through comparison of such systems with some aspects of the present disclosure, as set forth in the remainder of the present application with reference to the drawings.

### BRIEF SUMMARY

System and methods are provided for form-in-place shape-stabilized phase change material for transient cooling, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF THE DRAWINGS

FIG. 1 is a block diagram illustrating an example medical imaging arrangement.

FIG. 2 is a block diagram illustrating an example medical imaging probe with heat management.

FIG. 3 is a block diagram illustrating an example single fabricated structure with heat spreader and phase change material (PCM) substructures.

FIG. 4 is a block diagram illustrating an example integrated structure based on combining of heat spreader and phase change material (PCM) structures.

### DETAILED DESCRIPTION

The following detailed description of certain embodiments in accordance with the present disclosure will be better understood when read in conjunction with the appended drawings. The present disclosure is directed to solutions related to use of phase change material (PCM). In particular, solutions in accordance with the present disclosure are directed to form-in-place shape-stabilized phase change material (PCM) for transient cooling, such as for use in medical systems or components thereof (e.g., probes or other scanning devices). In this regard, these solutions may allow for elimination of containment structures that may be used in conventional solutions. Such containment structures are typically required because when PCM heats up, it becomes liquid, and thus may leak (e.g., within the probes). Containment structures may typically be a vessel-like structure, comprising solid walls for preventing the PCM from leaking. As noted, proposed solutions allow for use of PCM without use of containment structures. This may be done by, e.g., use of structures and/or additives with suitable characteristics to ensure that the PCM remains trapped without needing to be contained in dedicated containment structures, with the PCM is formed in place in a manner that ensure that the PCM does not leak out of the PCM structure. In this regard, as used herein forming in place may be a process where liquid PCM (pure or in conjunction with other material, such as filler material and/or additives) may be poured into a structure and allowed to form a solid composite structure therein which will not melt (and thus not leak) even when the composite structure is heated beyond the melting point of PCM, particularly where the temperature does not exceed the melting point(s) of filler material (when used).

For example, the PCM structure may have porous characteristics (e.g., a sponge-like structure, scaffolding structure, mesh-like structure, etc.) such that so it is rigid/hard but with spaces/gaps internally where the PCM may be filed and formed-in-place. Further, the PCM structure may be made of same material as the heat spreader, or at least compatible material, to optimize bonding and/or operation (thermal characteristics). Use of such structures, particularly when the material used therefor is suitably selected, may ensure optimal bonding to the spreader.

The PCM may be used by itself (pure PCM) or with filler material/additive material to optimize performance. For example, in some embodiments, inexpensive filler material (e.g., low-density polyethylene (LDPE), high-density polyethylene (HDPE), expanded graphite, etc.) may be used in conjunction with the PCM, with the filler material being particularly suitable for ensuring the formed-in-place PCM is maintained within the structure even when heated. In some instances, the filler material may be used along with other additives adaptively selected to improve thermal performance.

The proposed solutions also remedies some issues relating to bonding/attaching the PCM structure to the heat spreader. In this regard, a simple PCM containment has no problems of bonding or thermal contact to a metallic heat spreader; however, a shape stabilized PCM, especially commercially available ones, may be difficult to bond/fasten and hence unreliable. In particular, the bonding may typically be done by gluing or fastening the containment structures, but both approaches have limitations—e.g., difficulty in gluing the containment structures, possibility is creating leaking opening when fastening, etc. Use of combined structures in accordance with the present disclosure may resolve some of the issues with existing solutions by: 1) allowing for use of container-less PCM (reduced assembly, reduced risk of leaks), and 2) improving adhesion/bonding of shape stabilized PCM and hence better thermal contact. Another advantage of the proposed solutions is that the PCM structure will be directly in contact with the heat spreader rather than being indirectly connected to it (e.g., when enclosed within containment structures, as done in conventional implementations).

The mechanical characteristics of the structure itself, particularly in conjunction with the use of filler material (or other additives) when used, also help maintain the PCM in place. For example, capillarity properties may help maintain the PCM within the structure. In this regard, the capillary properties required to shape stabilize the PCM may be primarily offered by the filler material—e.g., LDPE and HDPE. The mechanical structure such as a mesh-like structure serves to hold the shape stabilized PCM close to the spreader, like a Velcro. The combination of PCM and filler material (with or without other additives) is poured into the mechanical structure in liquid form and allowed to solidify. Once solidified, the shape stabilized PCM is held in place even when heated above the melting point of PCM. However, in some embodiments, the structure may hold the PCM in place without the need for a filler material, due to its capillary characteristics of the structure, which may be achieved when the structure fabricated conventionally or additively. For example, the mechanical structure may have a fine porous structure formed by sintering, additive processes etc. to form capillaries, in which case the PCM may be held within the pores without the need for the filler material.

Further, the PCM may be maintained in the structure because of differences in characteristics of the PCM and other filler material used in the PCM structures. The PCM may remain in the structure because of, for example, differences in melting characteristics/temperatures of the PCM to the filler material (when used). For example, PCM melts at lower temperature compared to certain filler material (e.g., 30-40° C. for the PCM compared to about ˜100° C. for HDPE/LDPE). Thus, when filling forming the PCM in place, the PCM and the filler material are both melted, then combined. Afterwards, during thermal operation, the PCM structure is heated only up to melting temperature of the PCM so that the filler material remains solid, and the PCM remains trapped within the filler material/structure.

While various embodiments of the present disclosure are illustrated and described with respect to and in conjunction with medical imaging systems or components thereof (e.g., an ultrasound probe), solutions in accordance with the present disclosure are limited to such implementations, and may be applied substantially in similar manner to other suitable systems, devices, or environments, such as in systems requiring thermally managing any heat dissipating (e.g., electronics, batteries, etc.), embedding structures similar to the ones described herein in walls and structures of systems that are exposed to temperature extremes or swings wherein the invention could help stabilize the system temperature passively, etc.

To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” as used in the context of ultrasound imaging is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

In addition, as used herein, the phrase “pixel” also includes embodiments where the data is represented by a “voxel.” Thus, both the terms “pixel” and “voxel” may be used interchangeably throughout this document.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

In various embodiments, processing to form images is performed in software, firmware, hardware, or a combination thereof. The processing may include use of beamforming.

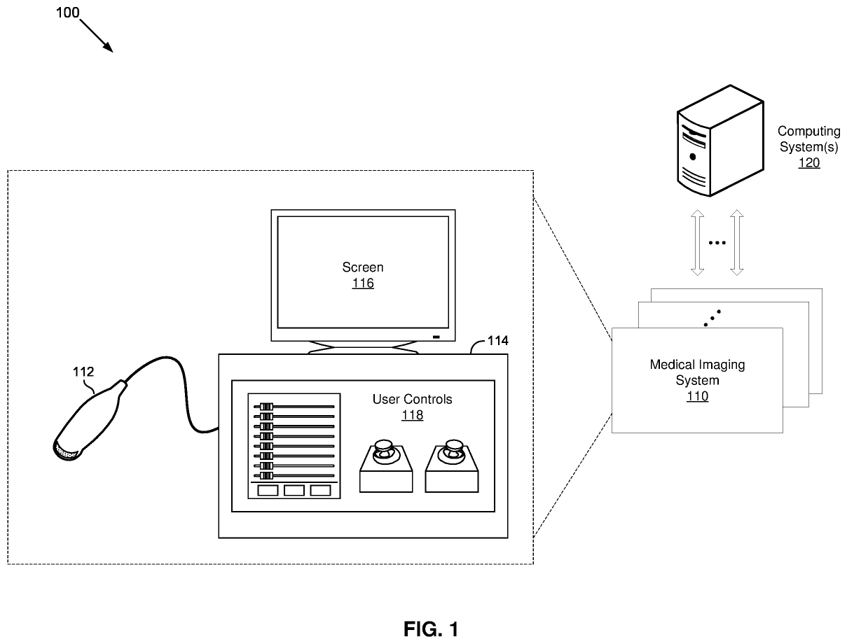

FIG. 1 is a block diagram illustrating an example medical imaging arrangement. Shown in FIG. 1 is an example medical imaging arrangement 100 that comprises one or more medical imaging systems 110 and one or more computing systems 120. The medical imaging arrangement 100 (including various elements thereof) may be configured to support medical imaging and solutions associated therewith.

The medical imaging system 110 comprise suitable hardware, software, or a combination thereof, for supporting medical imaging—that is enabling obtaining data used in generating and/or rendering images during medical imaging exams. Examples of medical imaging include ultrasound imaging, computed tomography (CT) scans, magnetic resonance imaging (MRI), etc. This may entail capturing of particular type of data, in particular manner, which may in turn be used in generating data for the images. For example, the medical imaging system 110 may be an ultrasound imaging system, configured for generating and/or rendering ultrasound images.

As shown in FIG. 1, the medical imaging system 110 may comprise a scanner device 112, which may be portable and movable, and a display/control unit 114. The scanner device 112 may be configured for generating and/or capturing particular type of imaging signals (and/or data corresponding thereto), such as by being moved over a patient's body (or part thereof), and may comprise suitable circuitry for performing and/or supporting such functions. The scanner device 112 may be an ultrasound probe, MRI scanner, CT scanner, or any suitable imaging device. For example, where the medical imaging system 110 is an ultrasound system, the scanner device 112 may emit ultrasound signals and capture echo ultrasound images.

The display/control unit 114 may be configured for displaying images (e.g., via a screen 116). In some instances, the display/control unit 114 may further be configured for generating the displayed images, at least partly. Further, the display/control unit 114 may also support user input/output. For example, the display/control unit 114 may provide (e.g., via the screen 116), in addition to the images, user feedback (e.g., information relating to the system, functions thereof, settings thereof, etc.). The display/control unit 114 may also support user input (e.g., via user controls 118), such as to allow controlling of the medical imaging. The user input may be directed to controlling display of images, selecting settings, specifying user preferences, requesting feedback, etc.

In some implementations, the medical imaging arrangement 100 may also incorporate additional and dedicated computing resources, such as the one or more computing systems 120. In this regard, each computing system 120 may comprise suitable circuitry, interfaces, logic, and/or code for processing, storing, and/or communication data. The computing system 120 may be dedicated equipment configured particularly for use in conjunction with medical imaging, or it may be a general purpose computing system (e.g., personal computer, server, etc.) set up and/or configured to perform the operations described hereinafter with respect to the computing system 120. The computing system 120 may be configured to support operations of the medical imaging systems 110, as described below. In this regard, various functions and/or operations may be offloaded from the imaging systems. This may be done to streamline and/or centralize certain aspects of the processing, to reduce cost—e.g., by obviating the need to increase processing resources in the imaging systems.

The computing systems 120 may be set up and/or arranged for use in different ways. For example, in some implementations a single computing system 120 may be used; in other implementations multiple computing systems 120, either configured to work together (e.g., based on distributed-processing configuration), or separately, with each computing system 120 being configured to handle particular aspects and/or functions, and/or to process data only for particular medical imaging systems 110. Further, in some implementations, the computing systems 120 may be local (e.g., co-located with one or more medical imaging systems 110, such within the same facility and/or same local network); in other implementations, the computing systems 120 may be remote and thus can only be accessed via remote connections (e.g., via the Internet or other available remote access techniques). In a particular implementation, the computing systems 120 may be configured in cloud-based manner, and may be accessed and/or used in substantially similar way that other cloud-based systems are accessed and used.

Once data is generated and/or configured in the computing system 120, the data may be copied and/or loaded into the medical imaging systems 110. This may be done in different ways. For example, the data may be loaded via directed connections or links between the medical imaging systems 110 and the computing system 120. In this regard, communications between the different elements in the medical imaging arrangement 100 may be done using available wired and/or wireless connections, and/or in accordance any suitable communication (and/or networking) standards or protocols. Alternatively, or additionally, the data may be loaded into the medical imaging systems 110 indirectly. For example, the data may be stored into suitable machine readable media (e.g., flash card, etc.), which are then used to load the data into the medical imaging systems 110 (on-site, such as by users of the systems (e.g., imaging clinicians) or authorized personnel), or the data may be downloaded into local communication-capable electronic devices (e.g., laptops, etc.), which are then used on-site (e.g., by users of the systems or authorized personnel) to upload the data into the medical imaging systems 110, via direct connections (e.g., USB connector, etc.).

In operation, the medical imaging system 110 may be used in generating and presenting (e.g., rendering or displaying) images during medical exams, and/or in supporting user input/output in conjunction therewith. The images may be 2D, 3D, and/or 4D images. The particular operations or functions performed in the medical imaging system 110 to facilitate the generating and/or presenting of images depends on the type of system—that is, the manner by which the data corresponding to the images is obtained and/or generated. For example, in computed tomography (CT) scans based imaging, the data is based on emitted and captured x-rays signals. In ultrasound imaging, the data is based on emitted and echo ultrasound signals.

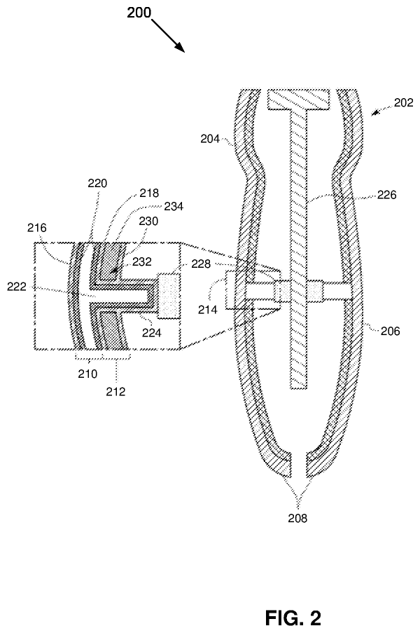

In various implementations in accordance with the present disclosure, medical imaging systems and/or architectures (e.g., the medical imaging system 110 and/or the medical imaging arrangement 100 as a whole) may be configured to support implementing and utilizing form-in-place shape-stabilized phase change material for transient cooling. This is described in more detail below, with respect to FIGS. 2-4.

FIG. 2 is a block diagram illustrating an example medical imaging probe with heat management. Shown in FIG. 2 is a medical imaging probe 200. The probe 200 may correspond to scanner device 112 of FIG. 1. In this regard, the probe 200 may be used in conjunction with medical imaging solutions, such as when obtaining signals or data related to imaging of a patient based on particular medical imaging technology. For example, the probe 200 may be an ultrasound probe in ultrasound based implementations.

In some instances, medical imaging systems, or components thereof (e.g., probes or other scanning devices) may incorporate heat thermal management related solutions. In this regard, an important factor that restricts the use of certain medical imaging techniques, such as ultrasound imaging, is that performing scanning requires extended operation of the probe (or scanning device in general) at high power, such as to render higher image resolution, while maintaining the surface and key component temperatures under their respective limits. Accordingly, in some instances, probes or other scanning devices may incorporate thermal management components that may be control the temperature of at least certain parts of the probes.

The probe 200 illustrated in FIG. 2 depicts an example implementation incorporating use of a three-dimensional (3D) phase change chamber that is configured to provide a thermal management structure. The phase change chamber may be in the form of a 3D vapor chamber (VC), a thermal energy storage chamber, or a combination thereof. The phase change chamber provides enhanced heat transport from internal heat generating components of the probe to an outer surface of the phase change chamber for cooling by the ambient environment and/or to phase change material (PCM) volumes for thermal energy absorption and storage. Additionally, the phase change chamber may also be configured to provide a mechanical support structure for the probe. The use of phase change chamber in probes, such as the probe 200, is described in more detail in incorporated U.S. patent application Ser. No. 16/212,863. As illustrated in FIG. 2, a phase change chamber of the probe 200 is a nested configuration of a 3D vapor chamber and a thermal energy storage chamber. Additionally, the 3D vapor chamber in the nested configuration includes a projection that is configured to be in thermal contact with a heat generating component of the probe 200.

The probe 200 includes a medical imaging probe handle 202. Also, the medical imaging probe handle 202 may include two or more segments such as a first segment 204 and a second segment 206 that are operatively coupled to one another. The probe 200 includes a thermal management assembly in the form of a phase change chamber 208 that is configured to provide enhanced thermal management for the probe 200, The phase change chamber 208 is monolithic with respect to a portion of the medical imaging probe handle 202 and is configured to thermally interface with one or more heat generating components in the probe 200 to dissipate the heat generated by the components of the probe 200. As depicted in FIG. 2, the phase change chamber 208 has a nested configuration. More particularly, the phase change chamber 208 includes a 3D vapor chamber 210 and a thermal energy storage chamber 212. An expanded view of a cross-section of one embodiment of the phase change chamber 208 is generally referenced by reference numeral 214.

The 3D vapor chamber 210 has hermetic chamber walls that extend around and define an enclosed chamber. The 3D vapor chamber 210 includes an external wall 216. Also, the phase change chamber 208 includes a common wall 218 that is shared by the 3D vapor chamber 210 and the thermal energy storage chamber 212. Additionally, the 3D vapor chamber 210 includes a working fluid that is disposed within a cavity between the external wall 216 and the common wall 218. The working fluid is configured to change phase in response to heat received from a component of the probe 200. Moreover, the 3D vapor chamber 210 includes a porous wick structure 220 configured to facilitate transport of the working fluid in the 3D vapor chamber 210. The porous wick structure 220 includes pores that are configured to hold the working fluid in a liquid phase in the 3D vapor chamber 210. Also, the porous wick structure 220 aids in returning the working fluid from the condenser end to the evaporator end of the 3D vapor chamber 210. Further, the 3D vapor change chamber 210 includes a vapor transport column or vapor space configured to aid in the transport of the working fluid in a vapor phase within the 3D vapor chamber 210.

In a presently contemplated configuration, one or more portions of the 3D vapor chamber 210 may extend inward from at least one of the hermetic chamber walls and at least partially towards an inner section of the medical imaging probe handle 202. This extension may be generally referred to as a projection 224. It may be noted that for ease of illustration the configuration of the 3D vapor chamber 210 of FIG. 2 is depicted as including one projection 224. However, the 3D vapor chamber 210 may include more than one projection 224. In this embodiment, the projection 224 of the 3D vapor chamber 210 is disposed in direct thermal contact with one or more components 226 of the probe 200 and configured to facilitate dissipation of heat generated by the components 226 of the probe 200. In certain embodiments, the probe 200 may also include a heat dissipating component 228. The heat dissipating component 228 is configured to thermally couple the 3D phase change chamber 210 to one or more heat generating components of the probe 200. Accordingly, in this example, the heat dissipating component 228 is positioned in direct thermal contact with one or more heat generating components 226 of the probe 200 and the projection 224 is thermally coupled to the heat dissipating component 228. Hence, the heat generated by the components 226 of the probe 200 is transferred to the projection 224 in the 3D vapor chamber 210 via the heat dissipating component 228.

Additionally, the phase change chamber 208 also includes the thermal energy storage chamber 212. The thermal energy storage chamber 212 has a hermetic chamber wall such as an internal wall 230. Also, a phase change material (PCM) 234 such as but not limited to organic types such as paraffin, inorganic PCM, eutectic alloys etc. is housed in a cavity 232 that is formed between the common wall 218 and the internal wall 230. Moreover, this phase change material (PCM) 234 is configured to change phase in response to heat received from a component 226 of the probe 200. The phase change material (PCM) 234 that is configured to transition between a solid phase and a liquid phase.

Further, for ease of illustration and description, the phase change chamber 208 is depicted as including two phase change chamber portions. These portions may be sealed to form the phase change chamber 208. Accordingly, in one embodiment, the phase change chamber 208 is a continuous structure.

The probe 200 including the medical imaging probe handle 202, the 3D vapor chamber 210 and thermal energy storage chamber 212 may be formed using additive manufacturing, such as by being formed using three-dimensional (3D) printing, rapid prototyping (RP), direct digital manufacturing (DDM), selective laser melting (SLM), electron beam melting (EBM), direct metal laser melting (DMLM), and the like. Some other exemplary methods of additive fabricating usable with the present specification may include processes, such as, but not limited to, direct writing, electron beam deposition, laser deposition, stereo-lithography, and the like.

The implementation illustrated in FIG. 2 represents an example of embodiment where phase change material (PCM) is maintained in a containment structure. In particular, as shown in FIG. 2 and described herein, the phase change material (PCM) 234 is maintained within the cavity 232 that is formed within a containment structure—e.g., comprising the common wall 218 and the internal wall 230, to ensure that the phase change material (PCM) does not leak when it becomes liquid. In solutions in accordance with the present disclosure, however, phase change material (PCM) may be utilized without requiring use of such containment structures. In this regard, installation of phase change material (PCM) for thermal energy storage requires filling and sealing inserts or chambers. This may have some disadvantages, however. In particular, such filling and sealing is an additional fabrication step. Further, phase change material (PCM) seal may be a risk over life of probe. Conventional shape-stabilized phase change material (PCM) based solutions, if any exist, may have drawbacks, such as unreliable adhesion to heat spreader or heat dissipating surfaces, making phase change material (PCM) less effective, and issues relating to use of mechanical fastening, which may lead to loss of phase change material (PCM).

Form-in-place Shape-stabilized phase change material (PCM) solutions in accordance with the present disclosure may overcome some of the issues, such as by using structures that may ensure reliable thermal connection with the heat dissipating surface, while eliminating the requirement of sealing and reducing the risk of PCM loss. For example, in some instances, phase change material (PCM) may be maintained within porous structure attached to heat dissipating surface or component, with additional measures that ensure that the PCM remains within the structure. The heat dissipating surface, may include, for example, an inner porous surface, which may be additively fabricated, sintered or incorporated therein by other suitable conventional methods, with the phase change material (PCM) filled into and formed within that porous structure. As noted, the phase change material (PCM) may be filled in and formed within the structure using measures to ensure that the PCM remains within the structure and not leak out even when the phase change material (PCM) melts during thermal operation.

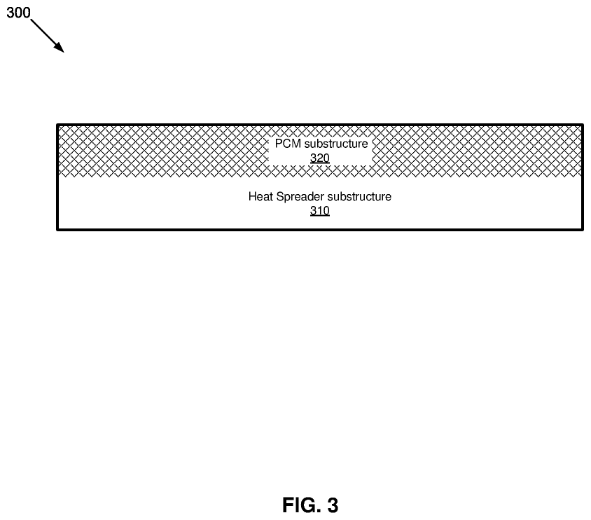

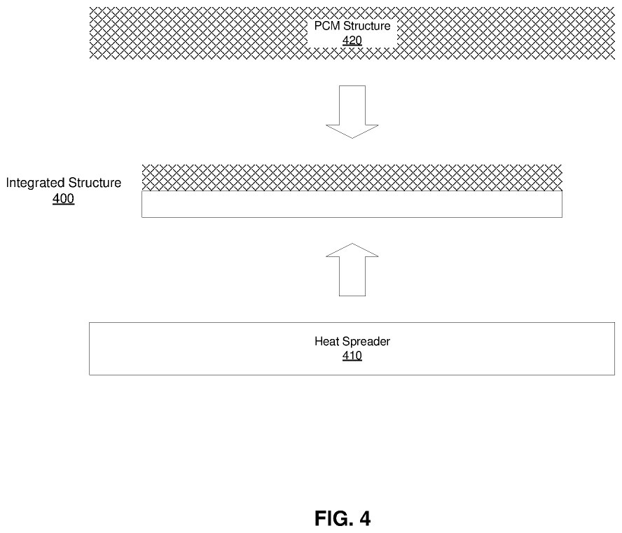

For example, in some implementations shape-stabilized PCM based mixture (e.g., comprising carrier material, such as low-density polyethylene (LDPE), high-density polyethylene (HDPE), and/or graphite, along with PCM, and (optionally) other additives) is used in liquid form at temperature higher than the melting point of the PCM is dispersed into the porous volume lining of the heated surface. When solidified in the porous volume, the PCM is held by capillarity property within the carrier and the porous conductive features of the structure hold the shape-stabilized PCM close to the heated surface, thus eliminating the risk of delamination. The shape-stabilized PCM could be formed-in-place directly in the heated surface, or could be fabricated on a separate surface which in-turn could be bonded to heated surface of the same material where adhesion risks are minimal. Various approaches and/or designed may be used in providing combined heater spreader and PCM structures. Example implementations are described in more detail below with respect to FIGS. 3 and 4.

While the implementation illustrated in FIG. 2 shows a 3D vapor chamber, the present disclosure is not limited to such arrangements, and as such in solutions in accordance with the present disclosure may be used in other embodiments without such arrangement. For example, some embodiments may include probes with heat spreader the inside of which has a thermo-mechanical structure (mesh, extended surfaces etc.) that serves to hold the shape stabilized PCM in thermal contact while offering mechanical support. In addition, where the structure is made out of a thermally conductive material (aluminum, copper, ceramic, etc.), the structure may enhance thermal performance of the PCM by reducing thermal resistance. Also, in the implementation illustrated in FIG. 2 the heat spreader is a 3D VC that is a monolithic structure with a PCM volume built-in internally. In accordance with the present disclosure, this monolithic structure may have extended surfaces instead of a containment to hold the shape stabilized PCM. Other variations may be used, however, including, for example: shape stabilized PCM formed directly as part of the probe heat spreader; 2) shape stabilized PCM with mechanical structure formed outside of the probe with a base substrate (same material as spreader or different material) that could be bonded or fastened to the internal spreader; and 3) the mechanical structure being fabricated using conventional or additive methods, to result in, e.g., a foam or mesh structure, extended surfaces such as pins, studs of different cross-sections, scaffolds of various cross-sections and designs, etc.

FIG. 3 is a block diagram illustrating an example single fabricated structure with heat spreader and phase change material (PCM) substructures. Shown in FIG. 3 is single fabricated structure 300. In this regard, a single fabricated structure may be made using single fabrication process configured to produce single fabricated structure that has a heat spreader (e.g., solid metal, such as aluminum) substructure that spreads the heat, and a substructure filled with phase change material (PCM). For example, as shown in FIG. 3, the single fabricated structure 300 may be fabricated as a structure comprising both of a heat spreader substructure 310 and a phase change material (PCM) substructure 320, with the PCM substructure 320 being configured to maintain formed-in-place PCM and to allow for its use in accordance with the present disclosure—that is, where the PCM may be heated (e.g., during heat management functions in medical imaging scanning devices) within the PCM substructure 320.

For example, the PCM substructure 320 may have porous characteristics (e.g., a sponge-like structure, scaffolding structure, mesh-like structure, etc.) such that so it is rigid/hard but with spaces/gaps internally where the PCM may be filled and formed-in-place. Further, the PCM substructure 320 may be made of same material as the heat spreader substructure 310, or at least compatible material, to optimize bonding and/or operation (thermal characteristics).

After fabrication, the PCM may be filled into the PCM substructure 320 and may form in place. In this regard, the PCM substructure 320 may comprise porous structure, and may specifically be configured to ensure that the formed-in-place PCM does not leak when heated. This may be done using the structure of the PCM substructure 320 itself, and/or by use of other additive filler material that would help ensure that the PCM remains within the structure, without requiring use of separate containment structures, as described herein.

In an example implementation, a coating layer may be applied to at least a portion of the exterior of the material structure. For example, one or more coatings may be applied over the PCM substructure 320, with the coating(s) acting as a secondary protection against leakage of the PCM when heated. Use of such coating(s) may be further enhance performance, particularly with respect to holding the PCM. For example, while in normal operation the capillary forces hold the PCM in place, such additional coating(s) may further protected against leakage under certain conditions, such as when the device comprising the fabricated structure 300 (e.g., an ultrasound probe) is subjected to abnormal movement (e.g., sudden acceleration, such as dropping or other conditions resulting in such sudden acceleration, which may result in the PCM leaking out of the substructure without the coating(s) being there help to contain the PCM).

FIG. 4 is a block diagram illustrating an example integrated structure based on combining of heat spreader and phase change material (PCM) structures. Shown in FIG. 4 an integrated structure 400. In this regard, for an integrated structure based approach, the heat spreader and phase change material (PCM) structures are fabricated separately, with the two structures subsequently attached to one another.

For example, in the example implementation shown in FIG. 4, the integrated structure 400 may be made using a heat spreader structure 410 and a phase change material (PCM) structure 420, which are combined together to form the integrated structure 400. The PCM may be filled into the PCM structure 420 and forms in place, with the filled PCM structure 420 subsequently attached (e.g., bonded) to the heat spreader structure 410. Alternatively, the (unfilled) PCM structure 420 may be attached to the heat spreader structure 410 first, and the PCM structure 420 is subsequently filled with PCM, with the PCM forming in place, after the two structures are integrated together. Further, as noted, pure PCM may be used, or a mixture of PCM and other additives or filler material (e.g., HDPE/LDPE) may be used.

The PCM structure—that is, the PCM structure 420 of the integrated structure 400—may be designed and/or configured to ensure that the PCM does not leak when heated. For example, as described above, the PCM structure 420 may have porous characteristics (e.g., a sponge-like structure, scaffolding structure, mesh-like structure, etc.) such that so it is rigid/hard but with spaces/gaps internally where the PCM may be filed and formed-in-place. Further, the PCM structure 420 may be made of same material as the heat spreader structure 410, or at least compatible material, to optimize bonding and/or operation (thermal characteristics). In an example implementation, one or more coating(s) may be applied to at least a portion of the exterior of the PCM structure 420, as described above with respect to FIG. 3.

An example combined structure, in accordance with the present disclosure, comprises: a heat spreading structure; and a material structure for holding phase change material; wherein: the material structure is arranged or disposed on a surface of the heat spreading structure; the phase change material is configured to change phase in response to heat from a component of a system comprising the combined structure; and the material structure has mechanical and structural characteristics for holding the phase change material within the material structure when the phase change material changes phase.

In an example implementation, the mechanical and structural characteristics comprise porous structural characteristics.

In an example implementation, the porous structural characteristics comprise one or more of sponge-like structure, scaffolding-like structure, and mesh-like structure.

In an example implementation, the phase change material is mixed with one or more filler material and/or additives for further assisting in holding the shape of phase change material within the material structure when the phase change material changes phase.

In an example implementation, the one or more filler material and/or additives comprise at least one of graphite, low-density polyethylene (LDPE), and high-density polyethylene (HDPE).

In an example implementation, the combined structure is fabricated as a singular and/or Monolithic structure with the heat spreading structure and the material structure being sub-structures thereof.

In an example implementation, the heat spreading structure and the material structure are fabricated separately and then attached to one another to create the combined structure.

In an example implementation, the phase change material or a mixture comprising the phase change material is filled into and forms in place within the material structure.

In an example implementation, the material structure and the heat spreading structure have substantially similar dimensions along the surface of the heat spreading structure where the material structure is disposed.

An example method, in accordance with the present disclosure, comprises: fabricating a combined structure for use in a system, wherein the combined structure comprises a heat spreading structure and a material structure for holding phase change material; wherein: the material structure is arranged or disposed on a surface of the heat spreading structure; the phase change material is configured to change phase in response to heat from a component of the system; and the material structure has mechanical and structural characteristics for holding the phase change material within the material structure when the phase change material changes phase.

In an example implementation, the method further comprises fabricating the combined structure as a singular structure, via a single fabrication process, with the heat spreading structure and the material structure being sub-structures of the singular structure.

In an example implementation, fabricating the combined structure comprising: separately fabricating each of the heat spreading structure and the material structure; and attaching the heat spreading structure and the material structure to one another to create the combined structure.

In an example implementation, the method further comprises filling the phase change material or a mixture comprising the phase change material into the material structure before attaching the material structure into the heat spreading structure, wherein the phase change material forms in place within the material structure.

In an example implementation, the method further comprises filling the phase change material or a mixture comprising the phase change material into the material structure after attaching the material structure into the heat spreading structure, wherein the phase change material forms in place within the material structure.

In an example implementation, the method further comprises selecting or configuring the material structure such that the mechanical and structural characteristics comprise porous structural characteristics.

In an example implementation, the porous structural characteristics comprise one or more of sponge-like structure, scaffolding-like structure, and mesh-like structure.

In an example implementation, the method further comprises mixing the phase change material with one or more filler material and/or additives, before filling the phase change material into the material structure, for further assisting in holding the phase change material within the material structure when the phase change material changes phase.

In an example implementation, the one or more filler material and/or additives comprise at least one of graphite, low-density polyethylene (LDPE), and high-density polyethylene (HDPE).

An example medical imaging system, in accordance with the present disclosure, comprises: a combined structure that comprises a heat spreading structure and a material structure for holding phase change material, wherein: the material structure is arranged or disposed on a surface of the heat spreading structure; the phase change material is configured to change phase in response to heat from a component of the medical imaging system; and the material structure has mechanical and structural characteristics for holding the phase change material within the material structure when the phase change material changes phase.

In an example implementation, the medical imaging system further comprises a probe or scanning device, and wherein the combined structure is used within the probe or scanning device.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments of the invention may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present invention may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims. What is claimed is:

1. A combined structure, comprising:
a heat spreading structure; and
a material structure for holding phase change material;
wherein:
the material structure is arranged or disposed on a surface of the heat spreading structure;
the phase change material is configured to change phase in response to heat from a component of a system comprising the combined structure; and
the material structure has mechanical and structural characteristics for holding the phase change material within the material structure when the phase change material changes phase.

2. The combined structure of claim 1, wherein the mechanical and structural characteristics comprise porous structural characteristics.

3. The combined structure of claim 2, wherein the porous structural characteristics comprise one or more of sponge-like structure, scaffolding-like structure, and mesh-like structure.

4. The combined structure of claim 1, wherein the phase change material is mixed with one or more filler material and/or additives for further assisting in holding the shape of phase change material within the material structure when the phase change material changes phase.

5. The combined structure of claim 4, wherein the one or more filler material and/or additives comprise at least one of graphite, low-density polyethylene (LDPE), and high-density polyethylene (HDPE).

6. The combined structure of claim 1, wherein the combined structure is fabricated as a singular and/or Monolithic structure with the heat spreading structure and the material structure being sub-structures thereof.

7. The combined structure of claim 1, wherein the heat spreading structure and the material structure are fabricated separately and then attached to one another to create the combined structure.

8. The combined structure of claim 1, wherein the phase change material or a mixture comprising the phase change material is filled into and forms in place within the material structure.

9. The combined structure of claim 1, wherein the material structure and the heat spreading structure have substantially similar dimensions along the surface of the heat spreading structure where the material structure is disposed.

10. A method, comprising:
fabricating a combined structure for use in a system, wherein the combined structure comprises a heat spreading structure and a material structure for holding phase change material;
wherein:
the material structure is arranged or disposed on a surface of the heat spreading structure;
the phase change material is configured to change phase in response to heat from a component of the system; and
the material structure has mechanical and structural characteristics for holding the phase change material within the material structure when the phase change material changes phase.

11. The method of claim 10, further comprising fabricating the combined structure as a singular structure, via a single fabrication process, with the heat spreading structure and the material structure being sub-structures of the singular structure.

12. The method of claim 10, wherein fabricating the combined structure comprising:
separately fabricating each of the heat spreading structure and the material structure; and
attaching the heat spreading structure and the material structure to one another to create the combined structure.

13. The method of claim 12, further comprising filling the phase change material or a mixture comprising the phase change material into the material structure before attaching the material structure into the heat spreading structure, wherein the phase change material forms in place within the material structure.

14. The method of claim 12, further comprising filling the phase change material or a mixture comprising the phase change material into the material structure after attaching the material structure into the heat spreading structure, wherein the phase change material forms in place within the material structure.

15. The method of claim 10, further comprising selecting or configuring the material structure such that the mechanical and structural characteristics comprise porous structural characteristics.

16. The method of claim 15, wherein the porous structural characteristics comprise one or more of sponge-like structure, scaffolding-like structure, and mesh-like structure.

17. The method of claim 10, further comprising mixing the phase change material with one or more filler material and/or additives, before filling the phase change material into the material structure, for further assisting in holding the phase change material within the material structure when the phase change material changes phase.

18. The method of claim 17, wherein the one or more filler material and/or additives comprise at least one of graphite, low-density polyethylene (LDPE), and high-density polyethylene (HDPE).

19. A medical imaging system, comprising:
a combined structure that comprises a heat spreading structure and a material structure for holding phase change material, wherein:
the material structure is arranged or disposed on a surface of the heat spreading structure;
the phase change material is configured to change phase in response to heat from a component of the medical imaging system; and
the material structure has mechanical and structural characteristics for holding the phase change material within the material structure when the phase change material changes phase.

20. The medical imaging system of claim 19, further comprising a probe or scanning device, and wherein the combined structure is used within the probe or scanning device.
